(19) United States
(12) Patent Application Publication
(10) Pub. No.: US 2019/0336733 A1
(43) Pub. Date: Nov. 7, 2019

(54) METHOD OF APPLYING THE DRUG

(71) Applicant: OLYMPUS CORPORATION, Tokyo (JP)

(72) Inventor: Hiroyuki KOJO, Kokubunji-shi (JP)

(73) Assignee: OLYMPUS CORPORATION, Tokyo (JP)

(21) Appl. No.: 15/970,250

(22) Filed: May 3, 2018

(51) Int. Cl. A61M 25/10; A61B 1/233; A61M 25/00; A61B 1/005; A61B 1/00

(52) U.S. Cl. CPC A61M 25/10; A61B 1/233; A61M 25/0026; A61M 2210/0681; A61B 1/00154; A61M 2025/105; A61M 2025/1061; A61B 1/005

(57) ABSTRACT

A drug is applied to a wall portion of a body cavity through a drug administration sheath including a first lumen that supplies a drug and a second lumen that is provided with at least one balloon. The drug administration sheath is inserted through an ostium of the body cavity until a distal end of the sheath reaches a deep part of the body cavity. The drug supplied through the first lumen is applied to a mucosa in the body cavity, utilizing inflation and movement of the balloon provided in the second lumen inside the body cavity so as to spread the drug along the mucosa.

### BACKGROUND OF THE INVENTION

### 1. Field of the Invention

The present invention relates to a method of applying a drug to mucosa in a sinus.

### 2. Description of the Related Art

Generally, a treatment method of applying a drug to an affected area is known. If the affected area is an exposed part, such as skin, the drug can be applied by a finger or the like. However, for an area in a sinus, such as a paranasal sinus, which cannot be accessed by a finger, a drug application method of using a catheter including a balloon is proposed, as disclosed in the patent publication, WO2015/073953. Specifically, in the method disclosed in the patent publication, a drug, such as an antibiotic, is applied to a surface of the balloon in advance. The balloon is inflated after it is inserted into the paranasal sinus, so that the drug on the surface of the balloon is pressed against the mucosa of the sinus.

As another method, the drug may be introduced to fill the sinus. However, in this method, depending on the size of the paranasal sinus, a large part of the drug may not have any direct effect on the treatment. Furthermore, there is a risk that facial pain may be caused by a pressure of the drug. Therefore, it is desirable that the drug without an excess be uniformly applied to only the surface of the mucosa.

The present invention provides a drug application method for evenly applying a drug having a high adhesive property on a surface of mucosa in a body cavity.

### BRIEF SUMMARY OF THE INVENTION

According to an embodiment of the present invention, there is provided a method of applying a drug on a wall portion of a body cavity through one of: a drug administration sheath including a first lumen that supplies a drug and a second lumen that is arranged along the first lumen and provided with at least one balloon; and a drug administration sheath including a first lumen that supplies a drug and a second lumen containing a sheath passing through the first lumen and provided with at least one balloon, the method comprising: inserting the drug administration sheath through an ostium of the body cavity until a distal end of the balloon reaches a deep part of the body cavity; and applying the drug supplied through the first lumen by inflating and moving the balloon inside the body cavity so as to spread the drug along a mucosa in the body cavity.

Advantages of the invention will be set forth in the description which follows, and in part will be obvious from the description, or may be learned by practice of the invention. The advantages of the invention may be realized and obtained by means of the instrumentalities and combinations particularly pointed out hereinafter.

### BRIEF DESCRIPTION OF THE SEVERAL VIEWS OF THE DRAWINGS

The accompanying drawings, which are incorporated in and constitute a part of the specification, illustrate embodiments of the invention, and together with the general description given above and the detailed description of the embodiments given below, serve to explain the principles of the invention.

FIG. 1 is a schematic view showing a sheath for administering a drug to practice a method of applying a drug according to an embodiment of the present invention.

FIG. 2A is a view for explaining a first step of a first method of applying a drug.

FIG. 2B is a view for explaining a second step of the first method of applying a drug.

FIG. 2C is a view for explaining a third step of the first method of applying a drug.

FIG. 3 is a flowchart for explaining the first method of applying a drug.

FIG. 4A is a view for explaining a first step of a second method of applying a drug.

FIG. 4B is a view for explaining a second step of the second method of applying a drug.

FIG. 4C is a view for explaining a third step of the second method of applying a drug.

FIG. 5 is a flowchart for explaining the second method of applying a drug.

FIG. 6A is a view for explaining a first step of a third method of applying a drug.

FIG. 6B is a view for explaining a second step of the third method of applying a drug.

FIG. 6C is a view for explaining a third step of the third method of applying a drug.

FIG. 6D is a view for explaining a fourth step of the third method of applying a drug.

FIG. 7 is a flowchart for explaining the third method of applying a drug.

FIG. 8 is a view for explaining a fourth method of applying a drug.

FIG. 9 is a flowchart for explaining the fourth method of applying a drug.

FIG. 10A is a view for explaining a first step of a fifth method of applying a drug.

FIG. 10B is a view for explaining a second step of the fifth method of applying a drug.

FIG. 10C is a view for explaining a third step of the fifth method of applying a drug.

FIG. 11 is a flowchart for explaining the fifth method of applying a drug.

FIG. 12 is a view showing a modification of a shape of an inflated balloon attached to a sheath for administering a drug for use in the fifth method of applying a drug.

FIG. 13A is a view for explaining a first step of a sixth method of applying a drug.

FIG. 13B is a view for explaining a second step of the sixth method of applying a drug.

FIG. 13C is a view for explaining a third step of the sixth method of applying a drug.

FIG. 13D is a view for explaining a fourth step of the sixth method of applying a drug.

FIG. 14 is a flowchart for explaining the sixth method of applying a drug.

FIG. 15 is a view showing a modification of a balloon of a sheath for administering a drug for use in the embodiment of the present invention.

FIG. 16A is a view showing a first placement in which a distal end of a balloon almost coincides with a distal end of a sheath for administering a drug.

FIG. 16B is a view showing a second placement in which a proximal end of a balloon almost coincides with a distal end of a sheath for administering a drug, so that the balloon having an annular shape is attached to the distal end of the sheath for administering a drug.

FIG. 16C is a view showing a third placement in which a balloon is placed in a portion of a sheath for administering a drug and spaced apart from the distal end.

### DETAILED DESCRIPTION OF THE INVENTION

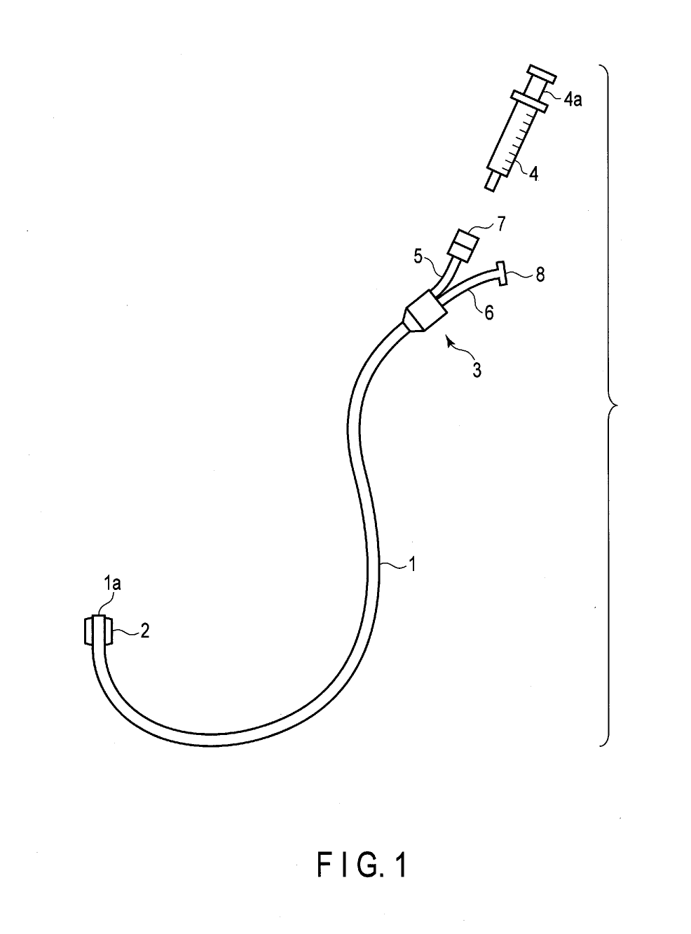

A method of applying a drug according to an embodiment of the present invention will be described below with reference to the accompanying drawings. FIG. 1 is a schematic view showing a sheath for administering a drug (or a catheter for administering a drug) to practice a method of applying a drug according to the embodiment of the present invention.

A sheath for administering a drug (hereinafter referred to as a drug administration sheath) 1 is a flexible tube. The drug administration sheath 1 includes at least two lumens 5 and 6 that integrally extend in a longitudinal direction, and a balloon 2 located near the distal end of the sheath. The lumen 5 is a drug administration lumen (a first lumen), which serves as an insertion passage to administer a drug. The other lumen 6 is a balloon lumen (a second lumen), which passes (supplies and discharges) a fluid to inflate or deflate the balloon 2. In this example, a distal end (opening) 5a of the drug administration lumen 5 and a distal end of the balloon 2 are both located at the same position in a distal end 1a of the drug administration sheath 1. In the following, two lumens are described as an example; however, the number of lumens is not limited to two, but the sheath may be provided with three or more lumens.

A proximal end of the drug administration sheath 1 is provided with a junction hub (hereinafter referred to as a hub) 3. The drug administration sheath 1 is divided into the drug administration lumen 5 and the balloon lumen 6 at the hub 3. A rear end of the drug administration lumen 5 is provided with an injection port 7, which is detachably connected to a syringe 4 filled with the drug. A rear end of the balloon lumen 6 is provided with a connector 8, which is to be connected to a fluid supply section (not shown).

As will be described later, a device for administration of a drug, for example, the syringe as shown in FIG. 1, can let a drug 11 flow into the drug administration lumen 5 by push-in of a cylinder 4a, and flow out through an opening of the distal end 5a of the lumen. Furthermore, the drug 11 can be sucked out of the sinus by pulling back the cylinder 4a of the syringe 4 in a state in which the opening of the distal end 5a of the drug administration lumen 5 is dipped in the drug, for example, in the recess of the paranasal sinus.

In this embodiment, the balloon 2 provided on the drug administration sheath 1 is inflated by a gas fluid or liquid fluid supplied thereto. The balloon 2 is not limited to a conventional spherical or bale-shape balloon, but may be shaped to inflate to a size (shape and volume) corresponding to a sinus of a target of treatment. If the target of treatment is, for example, a maxillary sinus 100 of the paranasal sinus as will be described later, the inflated shape of the balloon may be conical. The surface of the balloon 2 is water repellent, so that a gelatinous drug (to be described later) cannot be easily adhered thereto.

FIG. 16A, FIG. 16B, and FIG. 16C show examples of positions where the balloon is attached to the drug administration sheath. The balloon 2 of this embodiment is configured to cover all or part of the circumference of the drug administration sheath 1. Broadly speaking, the following three placements of the balloon may be considered: a first placement shown in FIG. 16A in which the distal end of the balloon 2 almost coincides with the distal end 5a of the drug administration lumen 5; a second placement shown in FIG. 16B in which the proximal end of the balloon 2 almost coincides with the distal end (opening) 5a of the drug administration lumen 5, so that the balloon having an annular shape is attached to the distal end (opening) of the drug administration lumen 5; and a third placement in which the balloon 2 shown in FIG. 16C is placed in a portion of the drug administration lumen 5 and spaced apart from the distal end (opening) the drug administration lumen 5. In the second placement, the drug is discharged after passing through the drug administration lumen 5 and the balloon 2. The third placement of the balloon is suitable for a case in which the patient is in a seating position, for example, in the case of treating the maxillary sinus. In this case, since the drug reaches the end of the sinus by the force of gravity, the balloon need not be placed at the distal end of the lumen. In the detailed description below, the drug is assumed to be injected from the distal end of the balloon 2 in the first and second placements of the balloon 2.

### [First Method of Applying Drug]

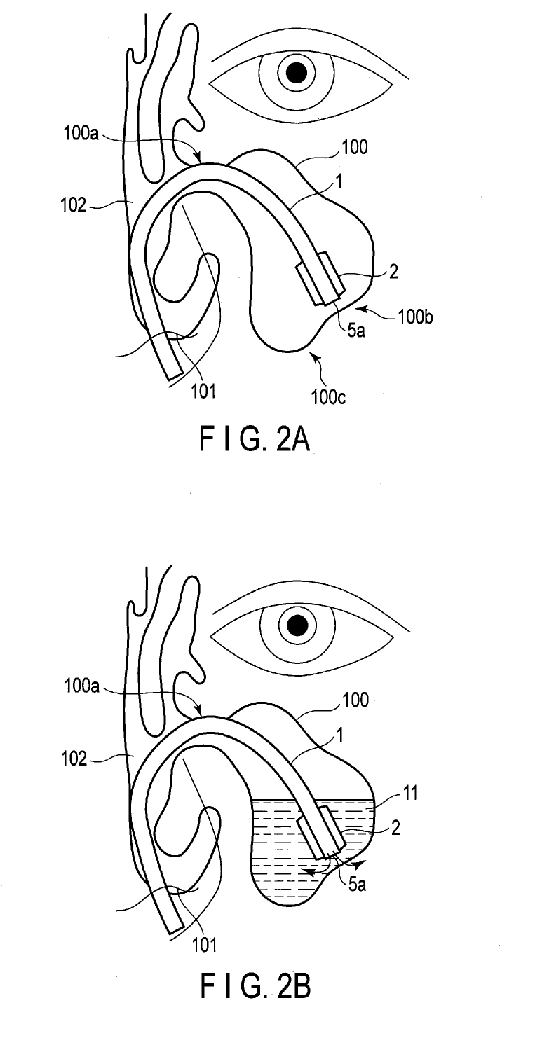

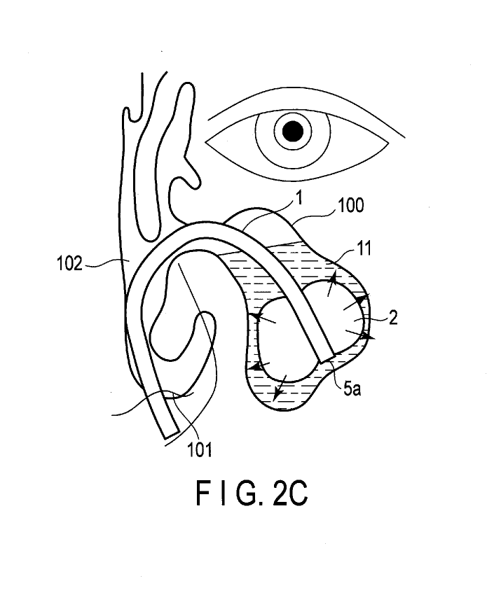

A first method of applying a drug will be explained with reference to FIG. 1, FIG. 2A, FIG. 2B, and FIG. 2C.

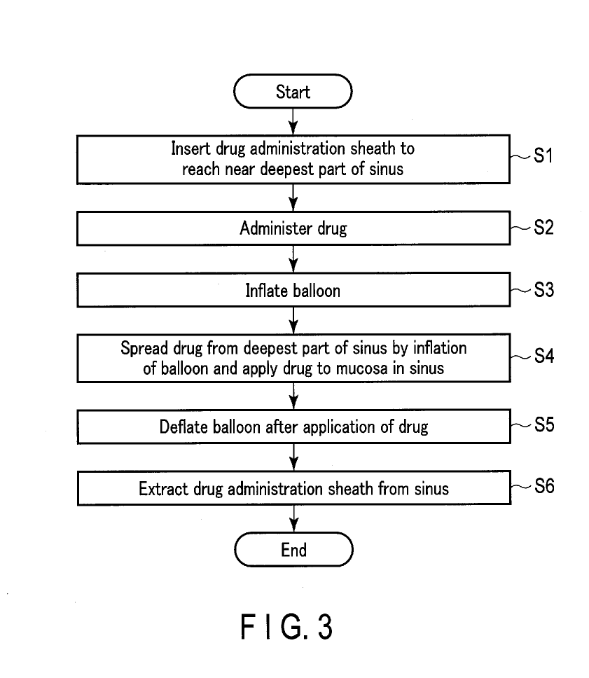

FIGS. 2A to 2C are views for explaining first to third steps of the first method of applying a drug. FIG. 3 is a flowchart for explaining the first method of applying a drug. In the methods of applying a drug described below, the target of application is an inner wall of a body cavity in general. As a specific example of the first method of applying the drug, mucosa in the maxillary sinus of the paranasal sinus will be explained. In this example, as in the case of the first placement shown in FIG. 16A, it is assumed that the distal end of the balloon 2 almost coincides with the distal end 1a of the drug administration sheath 1 and the drug is administrated through the distal end 5a of the balloon 2. Alternatively, the balloon 2 may be in the second placement described above.

As shown in FIG. 2A illustrating the first step, the maxillary sinus 100 is the largest part in the paranasal sinus, almost the same in shape as the maxilla, and has a nearly conical shape having a distal end projected toward a malar bone in a front view. In the explanation below, the maxillary sinus 100 has an ostium 100a in an inner topside of the sinus, and a deepest part (lateral/inferior) 100b in a lower side part of the sinus (or a part 100c most distant from the ostium 100a).

As shown in FIG. 2A, while observing via an endoscope (not shown), an operator inserts the drug administration sheath 1 into a nasal cavity 101, passing it through a nasal passage 102, until the sheath 1 reaches the ostium 100a of the maxillary sinus 100. Then, the operator inserts the distal end 1a of the drug administration sheath 1 through the ostium 100a, and moves it down to the lower side of the sinus, until the distal end 1a reaches around the deepest part 100b (step S1). The insertion into the maxillary sinus 100 is continued until the distal end 1a of the drug administration sheath 1 reaches the most distant part from the ostium of the sinus of the target of treatment or the lowermost part of the sinus in the direction of gravitational force.

Then, using the syringe 4 shown in FIG. 1, the drug 11 of a preset amount is administered into the maxillary sinus 100 through the drug administration lumen 5 (step S2), as shown in FIG. 2B illustrating the second step. The drug 11 is retained in the maxillary sinus 100, so that at least the balloon 2 (uninflated) is dipped in the drug 11. In the state in which the balloon 2 is dipped in the drug 11, the balloon 2 may not be completely dipped in the drug 11. When administering the drug 11, the drug administration sheath 1, in which the drug administration lumen 5 is filled with the drug 11 in advance, is inserted into the ostium of the target of treatment, so that the time to inject the drug 11 can be reduced. It is preferable that the drug 11 is a gelatinous material having an adhesive property higher than that of water, so that the adhesion state can be maintained when the drug is applied to the mucosa in the sinus. The administered amount of the drug 11 is an amount set in accordance with the size (volume) of the sinus to be treated.

After administering the drug 11, the balloon 2 is inflated by the gas fluid or liquid fluid supplied through the balloon lumen 6 (step S3), as shown in FIG. 2C illustrating the third step. As the balloon 2 inflates, the drug 11 in the maxillary sinus 100 is spread on the mucosa in the sinus from the deepest part toward the ostium, and evenly applied to the mucosa to reach the ostium 100a in an appropriate thickness (step S4). At this time, if the preset amount of the administered drug 11 is appropriate, the drug 11 is applied to the mucosa of the maxillary sinus 100 without an excess. However, when the drug 11 has been applied to reach the ostium 100a, if there is an excess of the drug 11, the excess will flow out of the ostium 100a into the nasal passage 102 and discharged to the outside.

After the application of the drug 11 to the mucosa of the maxillary sinus 100 is completed, the fluid in the balloon 2 is sucked to deflate the balloon (step S5). Then the drug administration sheath 1 is extracted from the maxillary sinus 100, and removed out of the body (step S6).

If the inflated balloon 2 does not cause the drug 11 to entirely cover the inside of the maxillary sinus 100, the drug administration sheath 1 may be pushed or pulled from the outside to move the inflated balloon 2 in the maxillary sinus 100 forward and backward (or up and down), so that the drug 11 can be entirely applied to the mucosa in the maxillary sinus 100. In this time, the excess of the drug 11, which was not applied to the mucosa, returns to the deepest part 100b via the wall surface in the sinus in the direction of gravitational force. The balloon 2 is deflated and the distal end 1a of the drug administration sheath 1 is extended the opening of the distal end 1a in the excess of the drug 11. The excess of the drug 11 may be sucked through the drug administration lumen 5 by operating the syringe 4 and discharged out of the maxillary sinus 100.

As described above, according to the first method of applying the drug, the gelatinous drug 11 can be spread and evenly applied to the mucosa from the deepest part of the maxillary sinus 100, utilizing the inflation of the balloon 2. Furthermore, by moving the inflated balloon 2 from the deepest part of the maxillary sinus 100 toward the ostium, the excess of the drug 11, which has not been used for the application, can be discharged from the ostium 100a of the maxillary sinus 100. The drug 11 may also be discharged from the ostium 100a of the maxillary sinus 100 by suction through the drug administration lumen 5.

In comparison with the conventional treatment for filling the sinus with the drug, the method of application of the drug can reduce the amount of the drug to be administered. Moreover, the risk that facial pain may be caused by a pressure of the drug 11 can be eliminated.

If the ostium 100a is located in an upper portion in the direction of gravity of the sinus, the excess of the drug cannot be naturally discharged against the gravitational force and remains in the sinus. Furthermore, since the gelatinous drug has a viscosity higher than that of water, it may continuously adhere to the inside of the sinus and cannot be discharged out of the sinus only by directing the ostium downward, depending on the shape of the sinus.

In contrast, according to the application method of this embodiment, while the drug is applied by utilizing the inflation of the balloon 2, the excess of the drug can be discharged out of the ostium 100a.

If the distal end 1a of the sheath is inserted into the gelatinous drug 11 after the drug 11 has been administered, a pushing force against the viscosity of the drug 11 will be required. However, since the drug administration sheath 1 is flexible, it is difficult to ensure the pushing force.

In contrast, according to the application method of this embodiment, the uninflated balloon 2 is first inserted into a deep part (lateral/inferior) of the maxillary sinus 100 and then the drug 11 is administered. The balloon 2, which is dipped in the gelatinous drug, is inflated. Therefore, the balloon 2 is inflated to raise the drug 11 from below, and spreads and applies the drug 11 onto the mucosa. Therefore, the application is easily performed without generating a force of pushing the drug administration sheath 1 into the drug 11.

### [Second Method of Applying Drug]

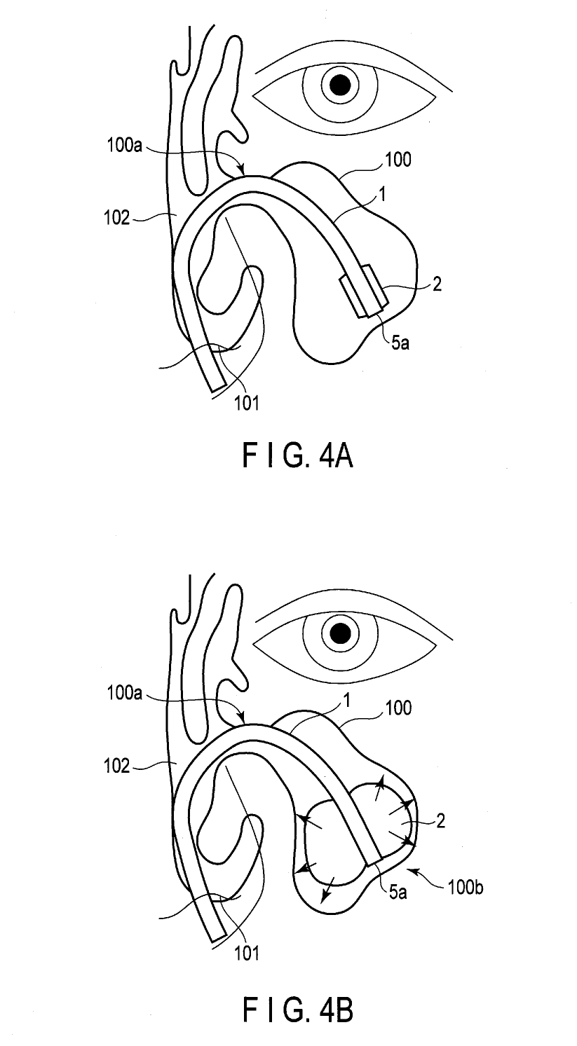

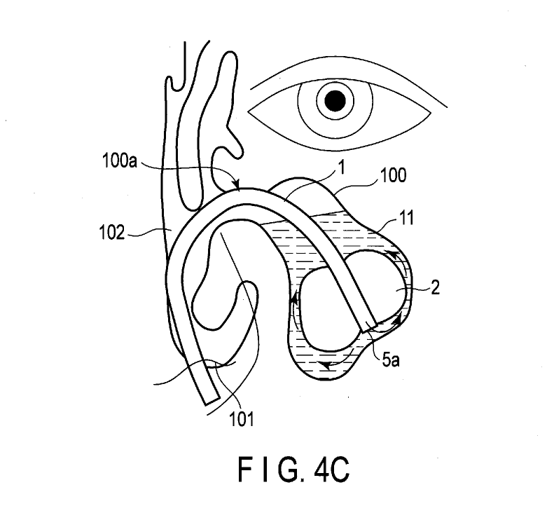

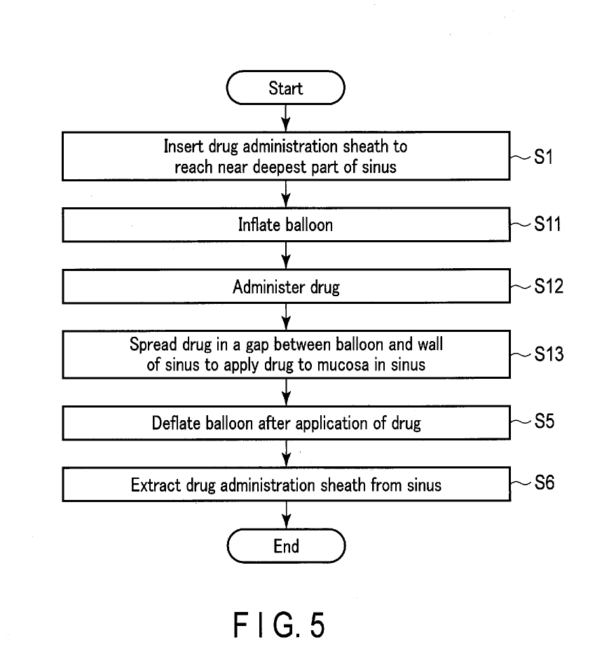

A second method of applying a drug will be explained with reference to FIG. 4A, FIG. 4B, and FIG. 4C. FIGS. 4A to 4C are views for explaining first to third steps of the second method of applying the drug 11. FIG. 5 is a flowchart for explaining the second method of applying a drug. As an example of the second method of applying the drug, the maxillary sinus of the paranasal sinus will be explained as a target of application.

In the first method of applying the drug described above, the balloon 2 is inflated after the drug 11 has been administered. However, in the second method of applying the drug, the drug 11 is administered after the balloon 2 has been inflated. In the method of applying the drug described below, the same numbers as those used in the description of the first method are used for the same method or the same step, and the explanations thereof are simplified.

As shown in FIG. 4A illustrating the first step, while observing via an endoscope (not shown), an operator inserts the drug administration sheath 1 into the nasal cavity 101, passing it through the nasal passage 102, until the sheath 1 reaches around the deepest part 100b of the maxillary sinus 100 (step S1). Then, the balloon 2 is inflated by the gas fluid or liquid fluid supplied through the balloon lumen 6 to fill the sinus in the maxillary sinus 100 (step S11), as shown in FIG. 4B illustrating the second step.

Furthermore, using the syringe 4 described above, the drug 11 of a preset amount is administered into the maxillary sinus 100 through the drug administration lumen 5 (step S12), as shown in FIG. 4C illustrating the third step. The gelatinous drug 11 is continuously administered and spread in the gap between the mucosa in the sinus and the inflated balloon 2, and evenly applied to the mucosa from the deepest part 100b to the ostium 100a in an appropriate thickness (step S13). When the drug 11 has been applied to reach the ostium 100a, if there is an excess of the drug 11, the excess will flow out of the ostium 100a into the nasal passage 102 and discharged to the outside.

After the application of the drug 11 to the mucosa in the maxillary sinus 100 is completed, the fluid in the balloon 2 is sucked to deflate the balloon 2 (step S5). Then, the drug administration sheath 1 is extracted from the maxillary sinus 100, and removed out of the body (step S6).

If the inflated balloon 2 has a size that does not cause the drug 11 to entirely cover the inside of the maxillary sinus 100, the balloon 2 may be moved in the same manner as in the first method of applying the drug, so that the drug 11 can be entirely applied to the mucosa in the maxillary sinus 100. Furthermore, the excess of the drug 11 may be sucked and discharged through the opening of the distal end 1a of the sheath out of the maxillary sinus 100.

As described above, according to the second method of applying the drug, the same effects and advantages as those of the first method of applying the drug can be obtained. Moreover, since the gelatinous drug is administered after the balloon 2 is inflated in the maxillary sinus 100 and the drug 11 is spread in the gap between the mucosa in the maxillary sinus 100 and the balloon 2, the inflating pressure of the balloon 2 can be lower than that of the balloon 2 in the method of spreading the drug 11 with the pressure of the balloon.

### [Third Method of Applying Drug]

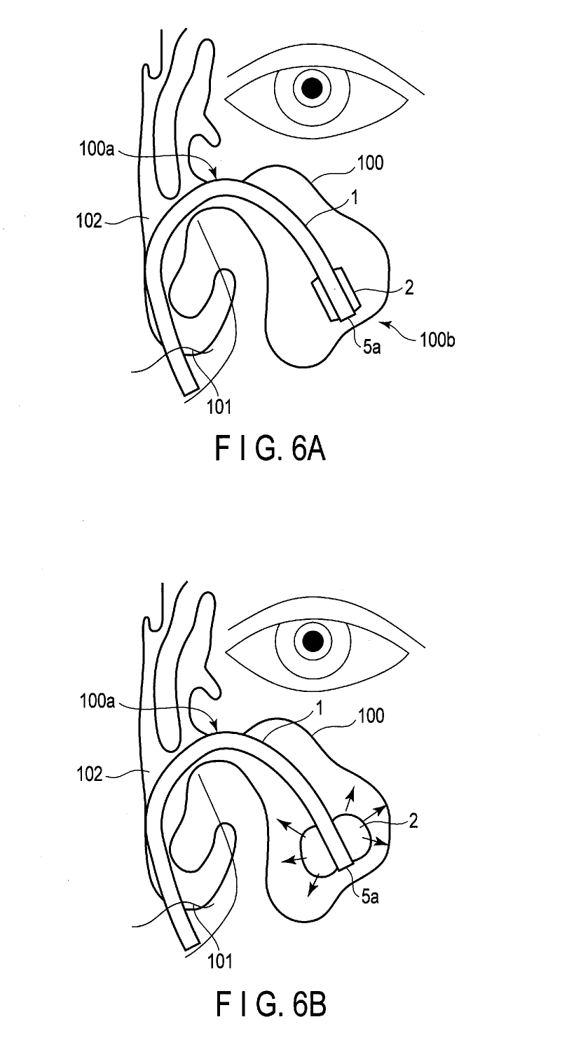

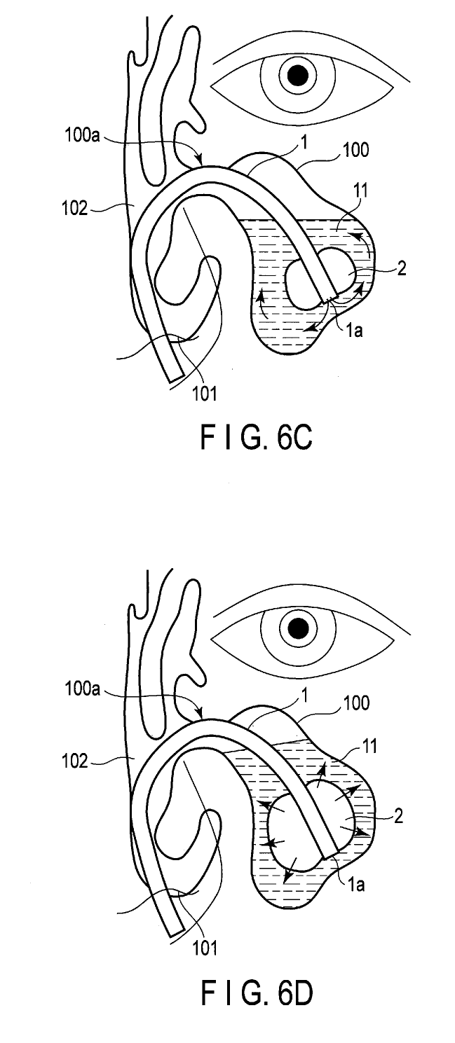

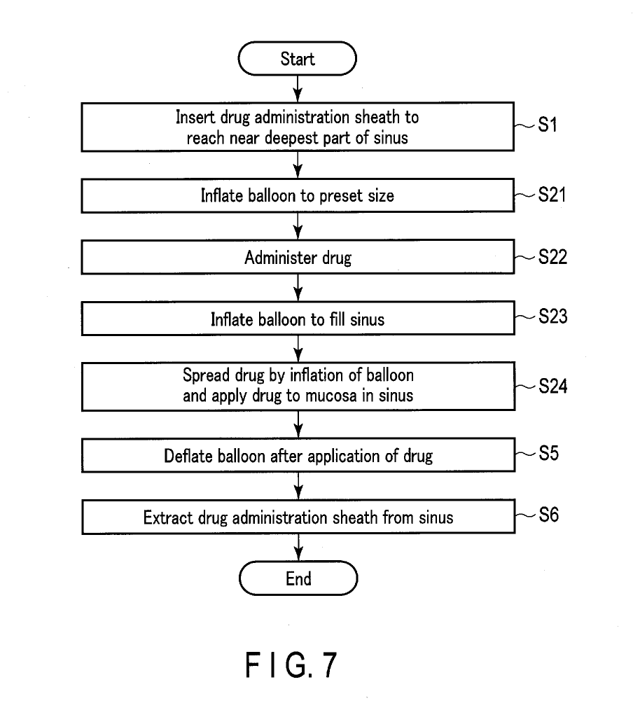

A third method of applying a drug will be explained with reference to FIG. 6A, FIG. 6B, FIG. 6C and FIG. 6D. FIG. 6A to 6D are views for explaining first to fourth steps of the third method of applying the drug 11. FIG. 7 is a flowchart for explaining the third method of applying a drug. As an example of the third method of applying the drug, the maxillary sinus of the paranasal sinus will be explained as a target of application.

In the third method of applying the drug, the balloon 2 is inflated in two steps, and the drug 11 is administered between the two steps of inflation to apply the drug 11 on the mucosa of the maxillary sinus 100. In the method of applying the drug described below, the same numbers as those used in the description of the first method are used for the same method or the same step, and the explanations thereof are simplified.

First, as shown in FIG. 6A illustrating the first step, while observing via an endoscope not shown, an operator inserts the drug administration sheath 1 into the nasal cavity 101, passing it through the nasal passage 102, until the sheath 1 reaches around the deepest part 100b of the maxillary sinus 100 (step S1).

Then, the balloon 2 is inflated to a preset size by the gas fluid or liquid fluid supplied through the balloon lumen 6 (see FIG. 1), as shown in FIG. 6B illustrating the second step (step S21). After the balloon 2 has been inflated to the preset size, the drug 11 of a preset amount is administered using the syringe 4 into the maxillary sinus 100 through the drug administration lumen 5 (see FIG. 1), as shown in FIG. 6C illustrating the third step (step S22). In other words, the drug 11 is administered on the balloon 2 that has been inflated in the maxillary sinus 100. Some of the drug 11 administered in this step flows to a part below the inflated balloon 2, spreads on the mucosa in the deepest part of the maxillary sinus 100, and the balloon 2 is immersed in the drug 11.

Then, the balloon 2 is inflated by the gas fluid or liquid fluid supplied through the balloon lumen 6 to fill the maxillary sinus 100 (step S23), as shown in FIG. 6D illustrating the fourth step. As the balloon 2 inflates, the gelatinous drug 11 is spread on the mucosa in the maxillary sinus 100, and evenly applied to the mucosa to reach the ostium 100a in an appropriate thickness (step S24). When the drug 11 has been applied to reach the ostium 100a, if there is an excess of the drug 11, the excess will flow out of the ostium 100a into the nasal passage 102 and discharged to the outside.

After the application of the drug 11 to the mucosa in the maxillary sinus 100 is completed, the fluid in the balloon 2 is sucked to deflate the balloon 2 (step S5). Then, the drug administration sheath 1 is extracted from the maxillary sinus 100, and removed out of the body (step S6).

If the inflated balloon 2 has a size that does not cause the drug 11 to entirely cover the inside of the maxillary sinus 100, the balloon 2 may be moved in the same manner as in the first method of applying the drug, so that the drug 11 can be entirely applied to the mucosa in the maxillary sinus 100. Furthermore, the excess of the drug 11 may be sucked and discharge through the opening of the distal end 1a of the sheath out of the maxillary sinus 100.

As described above, according to the third method of applying the drug, the same effects and advantages as those of the first method of applying the drug can be obtained. In this method, the gelatinous drug 11 is administered after the drug administration sheath 1 is inserted in the maxillary sinus 100. Thus, in comparison with the case in which the distal end 1a of the sheath is inserted in the drug 11 administered previously, a pushing force required for insertion against the viscosity of the drug 11 can be smaller. Moreover, since the drug 11 is administered and spread in the gap between the mucosa in the maxillary sinus 100 and the balloon 2 after the balloon 2 is inflated in the maxillary sinus 100, the inflating pressure of the balloon 2 can be lower than that of the balloon 2 in the method of spreading all the drug 11 with the pressure of the balloon.

### [Fourth Method of Applying Drug]

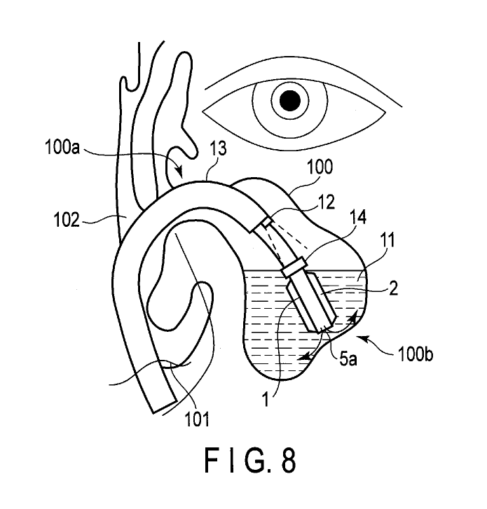

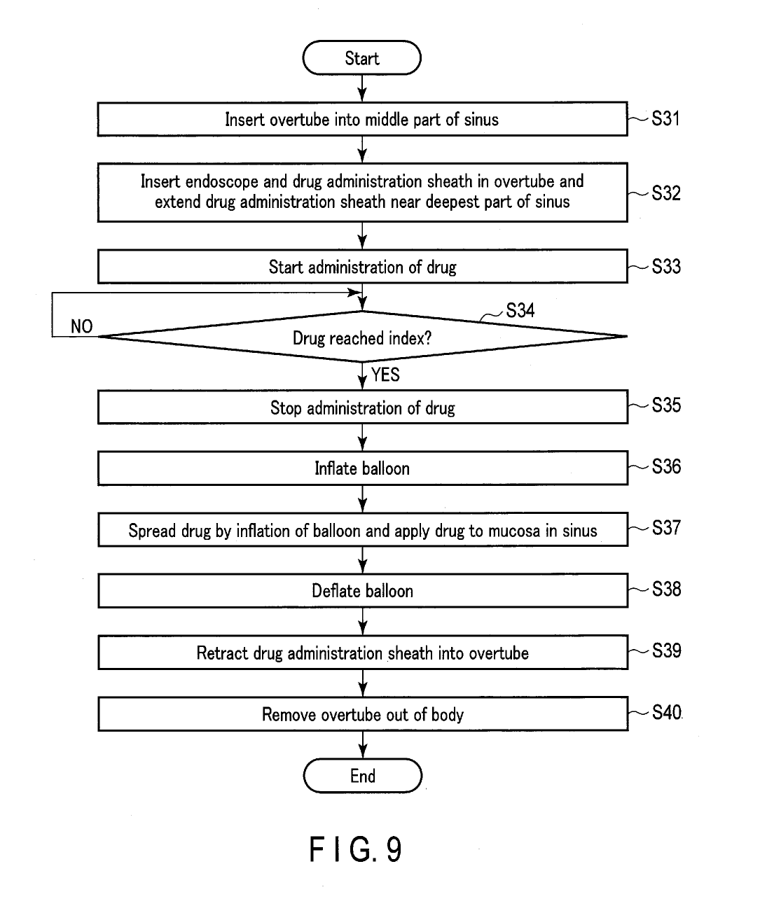

A fourth method of applying a drug will be explained with reference to FIG. 8. FIG. 8 is a view for explaining the fourth method of applying the drug to the maxillary sinus of the paranasal sinus as a target of application. FIG. 9 is a flowchart for explaining the fourth method of applying a drug. In the fourth method of applying the drug, the amount of drug 11 administered is controlled based on an index 14 on the drug administration sheath 1 to apply the drug to the mucosa in the maxillary sinus 100.

This method of application uses an overtube 13 including at least two lumens, through which a drug administration sheath 1 with a balloon 2 and an endoscope insertion section 12 are inserted.

A distal end portion of the drug administration sheath 1 is provided with at least one index 14 that indicates the amount of administration of the drug 11. The index 14 is belt-shaped and wrapped around the circumference of the drug administration sheath 1. The index 14 is formed of a member of a highly-visible color, a reflection member, or a light accumulation member, so that it can be easily recognized under dark illumination. The position where the index 14 is attached to the drug administration sheath 1 is determined based on the amount of administration of the drug 11. The index 14 is imaged by an imaging unit of the insertion section 12 of the endoscope inserted in the sinus together with the drug administration sheath 1, and displayed in a monitor or the like.

First, as shown in FIG. 8, an operator inserts the overtube 13 into the nasal cavity 101, passing it through the nasal passage 102, and through the ostium 100a of the maxillary sinus 100 to reach a middle part of the sinus (step S31). Next, the drug administration sheath 1 and the insertion section 12 of the endoscope are inserted from a proximal end of the overtube 13 and exposed out of a distal end of the overtube 13. Then, only the drug administration sheath 1 is extended until a distal end 5a of the sheath reaches around the deepest part 100b of the maxillary sinus 100 (step S32). At this time, the positional relationship (field range) between the insertion section 12 and the drug administration sheath 1 is adjusted, so that the distal end of the drug administration sheath 1 and the index 14 can be observed via the imaging unit of the insertion section 12.

Then, using the syringe 4 shown in FIG. 1, the operator starts administration of the drug 11 into the maxillary sinus 100 (step S33). After starting the administration, the operator observes the index 14 on the drug administration sheath 1 displayed by the imaging unit of the insertion section 12, and determines whether the administered drug 11 has reached the index 14 (step S34). In step S34, if it is determined that the drug 11 has not reached the index 14 (NO), the operator continues the administration. If it is determined that the drug 11 has reached the index 14 (YES), the operator stops the administration of the drug 11 by the syringe 4 shown in FIG. 1 (step S35).

After the administration of the drug 11 is completed, a gas fluid or a liquid fluid is supplied through the balloon lumen 6 (see FIG. 1) into the balloon 2 (step S36). As the balloon 2 inflates, the drug 11 in the maxillary sinus 100 is spread on the mucosa from the deepest part toward the ostium 100a, and evenly applied to the mucosa to reach the ostium 100a in an appropriate thickness (step S37). In this method of applying the drug, an appropriate amount of drug can be administered. However, depending on a change in the state of the maxillary sinus 100, there may be an excess of the drug 11. In such a case, the excess will flow out of the ostium 100a into the nasal passage 102 and discharged to the outside.

After the application of the drug 11 to the mucosa of the maxillary sinus 100 is completed, the fluid in the balloon 2 is sucked to deflate the balloon 2 (step S38). Then, the drug administration sheath 1 is retracted into the overtube 13 (step S39). At the same time, the insertion section 12 is also retracted into the overtube 13. Then, the overtube 13 is removed from the maxillary sinus 100 out of the body (step S40).

As described above, according to the fourth method of applying the drug, the same effects and advantages as those of the first method of applying the drug can be obtained. Furthermore, the amount of administered drug 11 can be controlled appropriately by the index that indicates the preset amount of administration of the drug. According to the above description of the fourth method of applying the drug, the drug 11 is administered after the drug administration sheath 1 with the uninflated balloon is inserted into the maxillary sinus 100, in the same manner as in the first method of applying the drug. However, only by changing the position of the index 14, the fourth method can be applied to the third method, in which the drug 11 is administered between the two steps of inflation of the balloon 2 to apply the drug 11 on the mucosa of the maxillary sinus 100.

The fourth method of applying the drug uses at least two lumens in the overtube 13, through which the drug administration sheath 1 with the balloon 2 is inserted. However, the embodiment is not limited to this configuration. For example, an endoscope with a working channel can be used in combination with the drug administration sheath 1. In this case, the drug administration sheath 1 is inserted through the working channel and the distal end 5a of the sheath reaches around the deepest part 100b in the maxillary sinus 100.

### [Fifth Method of Applying Drug]

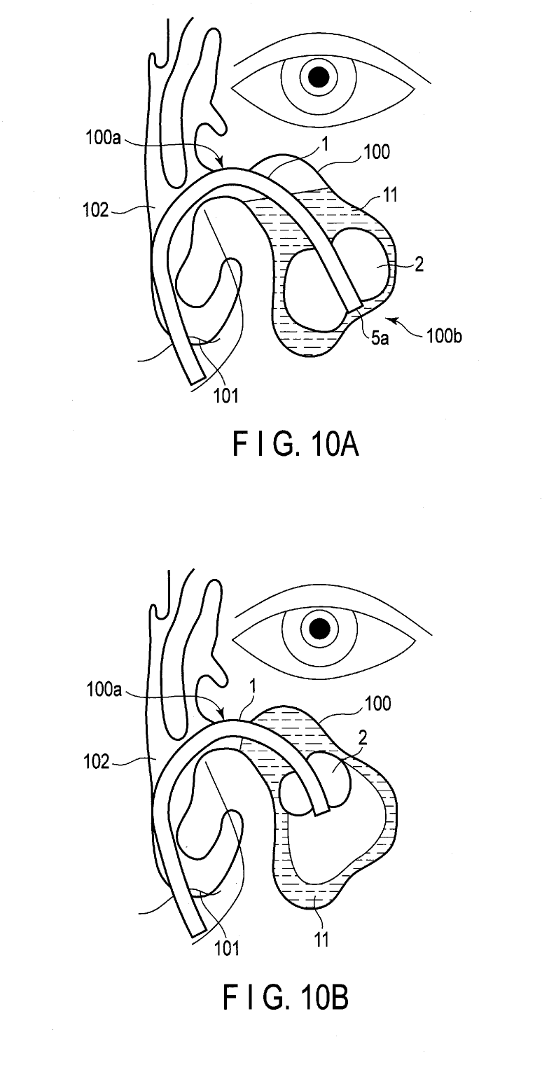

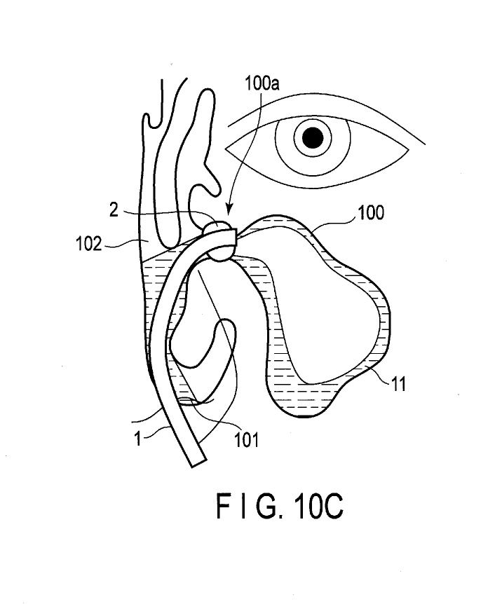

A fifth method of applying a drug will be explained with reference to FIG. 10A, FIG. 10B, and FIG. 10C.

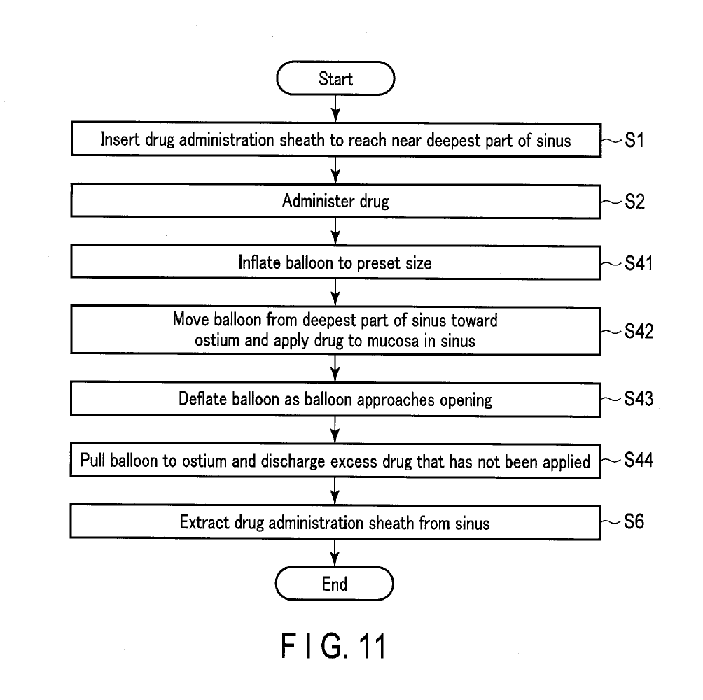

FIGS. 10A to 10C are views for explaining first to third steps of the fifth method of applying a drug. FIG. 11 is a flowchart for explaining the fifth method of applying a drug. As an example of the fifth method of applying the drug, the maxillary sinus of the paranasal sinus will be explained as a target of application. In the method of applying the drug described below, the same numbers as those used in the description of the first method are used for the same method or the same step, and the explanations thereof are simplified.

First, as shown in FIG. 10A, while observing via an endoscope not shown, an operator inserts the drug administration sheath 1 into the nasal cavity 101, passing it through the nasal passage 102, until the sheath 1 reaches around the deepest part 100b of the maxillary sinus 100 (step S1). Next, using the syringe 4 described above, the drug 11 of a preset amount is administered into the maxillary sinus 100 through the drug administration lumen 5 (see FIG. 1) (step S2).

Then, a gas fluid or a liquid fluid of a predetermined amount is supplied through the balloon lumen 6 (see FIG. 1) into the balloon 2 in the maxillary sinus 100 to inflate it to a preset size (step S41). The size of the inflated balloon is preset at least such that the outer diameter of the balloon 2 is approximate to the width of the deepest part of the maxillary sinus 100, and a gap is left between the balloon 2 and the mucosa in the maxillary sinus 100 to allow the drug 11 to be applied to the mucosa.

Next, as shown in FIG. 10B illustrating the second step, the drug administration sheath 1 is slowly pulled through the nasal passage 102 to move the balloon 2 toward the ostium 100a of the maxillary sinus 100. This movement causes the drug 11 to be spread and applied to the mucosa in the maxillary sinus 100 (step S42). As a result of this pulling movement, the drug 11 retained on the balloon 2 moves toward the ostium 100a along with the balloon 2. At this time, the drug 11 of the amount corresponding to the gap between the balloon 2 and the mucosa in the maxillary sinus 100 remains on the mucosa. Thus, the drug 11 is applied to the mucosa. Then, as shown in FIG. 10C illustrating the third step, the drug administration sheath 1 is pulled out. As the balloon 2 approaches the ostium, the fluid in the balloon moving from the deepest part toward the ostium 100a is gradually discharged to maintain a predetermined pressure in the balloon 2, so that the balloon is deflated to reduce the outer diameter of the balloon 2 (step S43). The deflation of the balloon 2 is based on the assumption that the width of the maxillary sinus 100 is reduced from the deepest part toward the ostium 100a. When the balloon 2 is pulled up to the ostium 100a of the maxillary sinus 100, the drug 11 that has not been applied to the mucosa in the maxillary sinus 100 is discharged out to the nasal passage 102 (step S44). Then, the drug administration sheath 1 is extracted from the maxillary sinus 100, and removed out of the body (step S6).

With the fifth method of applying the drug, the operation of removing the inflated balloon 2 from the maxillary sinus 100 through the ostium 100a causes the drug 11 to be evenly applied to the mucosa of the wall portion with the balloon 2 from the deepest part to the ostium of the maxillary sinus 100. The balloon 2 may be deflated by discharging the fluid from the balloon 2 appropriately as the sinus becomes narrower toward the ostium of the maxillary sinus 100 by controlling the pressure of the fluid in the balloon to be constant, when the balloon 2 moves toward the ostium. At this time, since the balloon 2 is deflated appropriately by flow out of the fluid, the effect of spreading the drug is maintained, while the balloon 2 is deflated in accordance with the narrowing of the sinus.

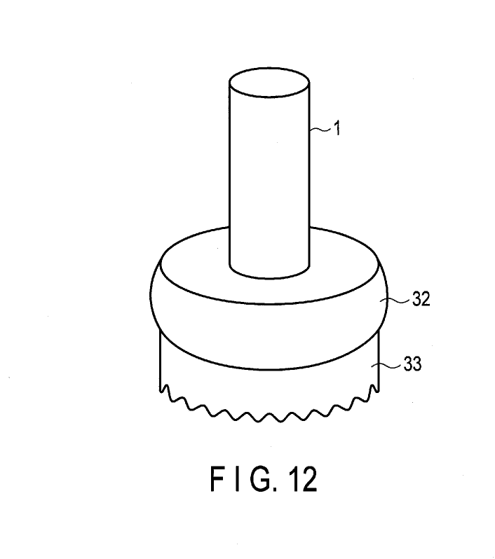

FIG. 12 is a view showing a modification of a shape of an inflated balloon attached to a sheath for administering a drug for use in the fifth method of applying a drug. The drug administration sheath 1 is provided with a balloon 32 radially inflated and expanded to a disk shape. The balloon 32, which is inflated to be a disk shape, can move in the sinus and apply the drug to the mucosa wall with a light load. A skirt 33 of a desired length is attached around the circumference of the balloon 32. When the balloon 32 moves in the sinus to be treated, the skirt 33 clings to the mucosa in the sinus and daubs the drug 11 over the mucosa while moving. As a result, the drug is applied to even recessed parts of the mucosa without fail to a more uniform thickness.

### [Sixth Method of Applying Drug]

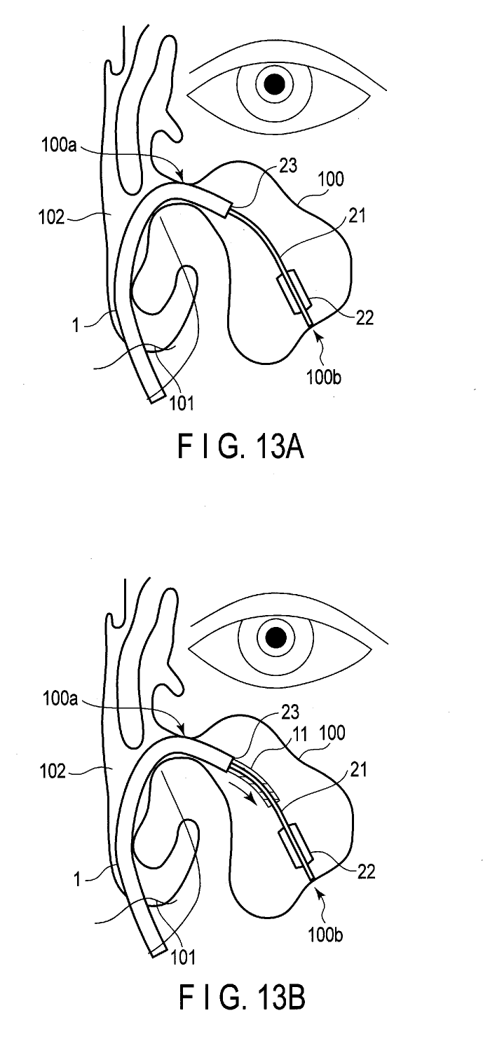

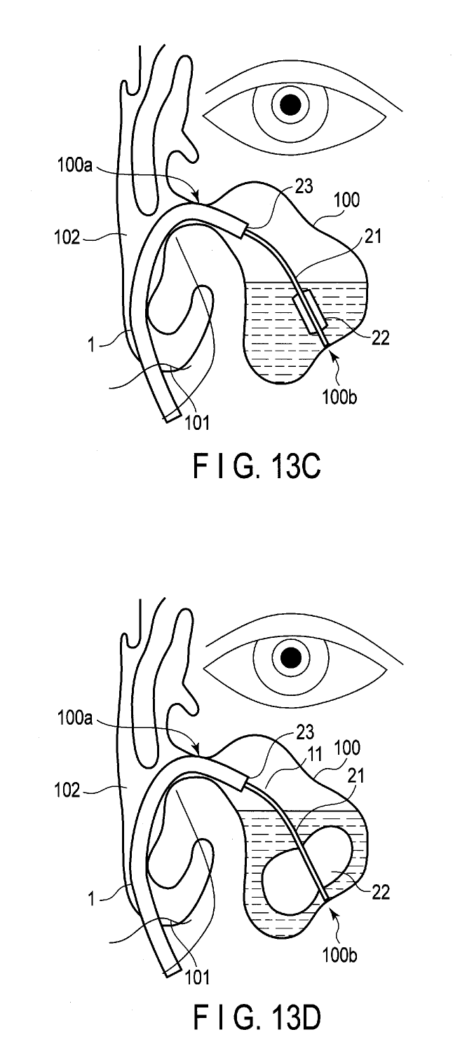

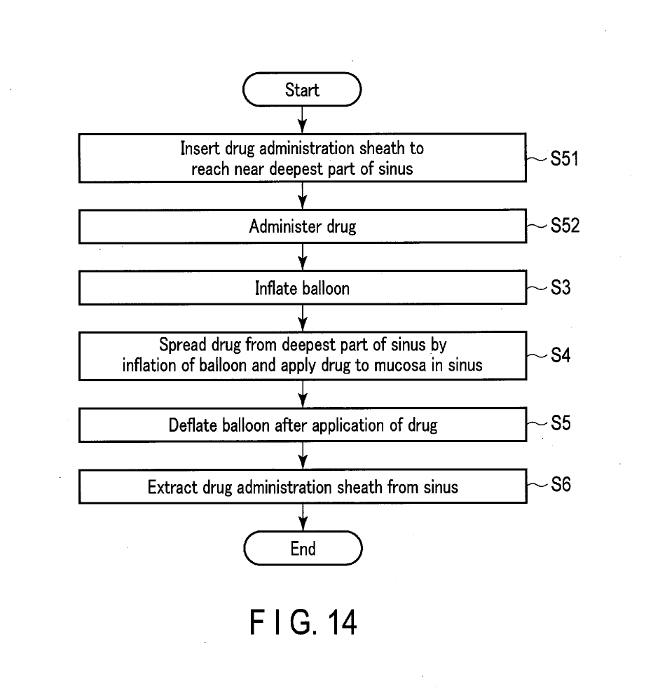

A sixth method of applying a drug will be explained with reference to FIG. 13A, FIG. 13B, FIG. 13C and FIG. 13D. FIGS. 13A to 13D are views for explaining first to fourth steps of the sixth method of applying the drug. FIG. 14 is a flowchart for explaining the sixth method of applying a drug. As an example of the sixth method of applying the drug, the maxillary sinus of the paranasal sinus will be explained as a target of application. In the method of applying the drug described below, the same numbers as those used in the description of the first method are used for the same method or the same step, and the explanations thereof are simplified. A drug administration sheath 1 for use in the sixth method of applying the drug functions as a drug administration lumen 23, which allows passage of a balloon sheath 21 provided with a balloon 22 and also allows administration of a drug. In other words, the drug administration sheath 1 is configured to administer the drug through the lumen that allows passage of the balloon sheath 21 as the drug administration lumen 23, and not to administer the drug through the balloon sheath 21. The balloon sheath 21 supplies gas or liquid to inflate the balloon 22.

As shown in FIG. 13A illustrating the first step, the drug administration sheath 1 is inserted into the maxillary sinus 100 (step S51). When the drug administration sheath 1 is inserted into the maxillary sinus 100, only the distal end of the balloon sheath 21 extends to a part near the deepest part 100b, so that the balloon 22 is present near the deepest part 100b. On the other hand, the drug administration lumen 23 is located near the ostium of the sinus.

As shown in FIG. 13B illustrating the second step, the drug 11 is administered into the maxillary sinus through the drug administration lumen 23 (step S52). In the first method of applying the drug shown in FIG. 2B, the opening of the drug administration lumen 23 is located near the inner wall of the maxillary sinus. Therefore, the drug 11 flowing straight out of the opening of the drug administration lumen 23 is first brought into contact with the inner wall of the maxillary sinus and then changes in direction along the surface of the inner wall.

In contrast, as shown in FIG. 13B illustrating the second step of the sixth method, the ostium of the drug administration lumen 23 is distant from the inner wall of the maxillary sinus. Thus, the drug 11 administered from the syringe 4 shown in FIG. 1 flows without disturbance by the inner wall, and collects in the bottom of the maxillary sinus.

Then, after administering the drug 11, the balloon 22 is inflated by the gas fluid or liquid fluid supplied through the balloon sheath 21 (step S3), as shown in FIG. 13C illustrating the third step. As the balloon 22 inflates, the drug 11 in the maxillary sinus 100 is spread on the mucosa from the deepest part toward the ostium 100a, and evenly applied to the mucosa to reach the ostium in an appropriate thickness (step S4). At this time, if the preset amount of the administered drug 11 is appropriate, the drug 11 is applied to the mucosa of the maxillary sinus 100 without an excess. However, when the drug 11 has been applied to reach the ostium 100a, if there is an excess of the drug 11, the excess will flow out of the ostium 100a into the nasal passage 102 and discharge to the outside.

After the application of the drug 11 to the mucosa of the maxillary sinus 100 is completed, the drug in the maxillary sinus 100 is sucked to deflate the balloon 22 (step S5). Then, the drug administration sheath 1 is extracted from the maxillary sinus 100, and removed out of the body (step S6).

According to the sixth method of applying the drug, the ostium of the drug administration lumen 23 is distant from the inner wall of the maxillary sinus 100. The drug 11 flows without disturbance by the inner wall, and flows straight out of the ostium. Therefore, in the case of administering the drug 11 by the syringe 4 (see FIG. 1), the load of pushing the cylinder 4a is reduced.

### [Modification of Balloon for Sheath for Administering Drug]

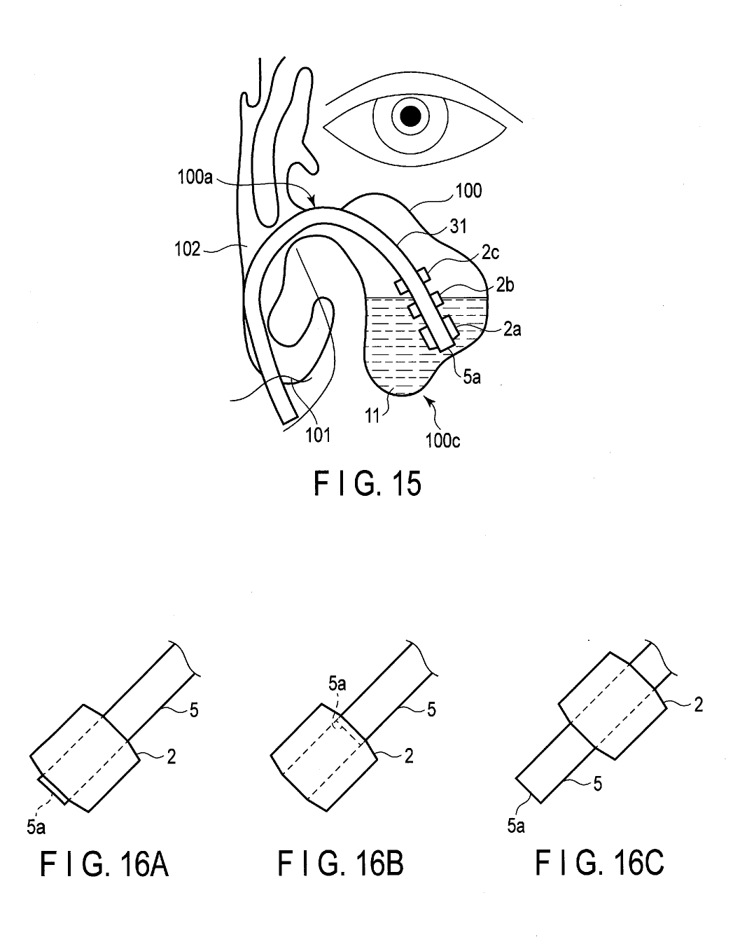

A modification of the balloon for a sheath for administering a drug (a drug administration sheath) will be described with reference to FIG. 15. FIG. 15 is a schematic view showing a drug administration sheath 31 provided with a plurality of balloons 2a to 2c. The sheath 31 for administering a drug, provided with a plurality of balloons, can be used for the first to sixth methods for applying the drug described above.

In the first to sixth methods of applying the drug, a configuration of using a single balloon 2 is described as an example. In this modification, the drug administration sheath 31 includes a plurality of balloon lumens (not shown) and provided with the three balloons 2a, 2b, and 2c, as shown in FIG. 15. The modification is applicable to a drug administration sheath with two or more balloons, although the three balloons are used in this example.

The drug administration sheath 31 used in the modification includes three balloon lumens (not shown) respectively provided with the balloons 2a, 2b, and 2c, as shown in FIG. 15. In the drug administration sheath 31, the positions of the balloon lumens are adjusted so that the balloons are arranged in series along the longitudinal direction of the sheath.

The balloons 2a, 2b, and 2c are individually allocated to divided areas in the sinus as a target of treatment along the longitudinal direction of the sheath. For example, if the target of treatment is a maxillary sinus 100, the distal end of the drug administration sheath 31 is inserted to the deepest part of the sinus, and the drug 11 is administered through an ostium of a distal end 1a of the sheath.

Thereafter, the balloon 2a of the most distal part is first inflated, then the balloons 2b and 2c are successively inflated with a time difference. The successive inflation of the balloons 2a, 2b, and 2c causes the drug 11 to spread and rise toward an ostium 100a.

All of the balloons 2a, 2b, and 2c are not necessarily inflated, and some of them may be independently inflated in accordance with the size or shape of the sinus. For example, in the case of treating a frontal sinus (not shown), if the patient is in a seating position, an ostium of the sinus is directed downward. In this case, depending on the viscosity of the drug, even if the drug is administered in a state where the distal end of the drug administration sheath 31 is inserted to the deepest part, the drug may flow out as streaks from the ostium along the sinus wall and cannot be uniformly applied to the mucosa wall.

To avoid this, the balloon 2c closest to the ostium is first inflated to a certain extent to function as a stopper, and then the drug 11 is administered. Thereafter, the balloon 2b and the balloon 2a are successively inflated in this order, while the balloon 2c is deflated, so that the drug can be applied to the mucosa of the frontal sinus.

In the above description of the methods for applying the drug, the drug is described as a gelatinous drug. However, the methods can be applied to a jelly drug in the same manner.

The present invention is not limited to the methods of applying the drug described above, but can be modified in various ways in practice without departing from the gist of the invention. The methods of applying the drug can be combined as appropriate if possible. In that case, combinational effects can be obtained. The methods of applying the drug described above involve various aspects of the invention, and appropriate combinations of the disclosed features permit various inventions to be derived.

1. A method of applying a drug on a wall portion of a body cavity through one of: a drug administration sheath including a first lumen that supplies a drug and a second lumen that is arranged along the first lumen and provided with at least one balloon; and a drug administration sheath including a first lumen that supplies a drug and a second lumen containing a sheath passing through the first lumen and provided with at least one balloon, the method comprising:
inserting the drug administration sheath through an ostium of the body cavity until a distal end of the balloon reaches a deep part of the body cavity; and
applying the drug supplied through the first lumen by inflating and moving the balloon inside the body cavity so as to spread the drug along a mucosa in the body cavity.

2. The method of applying a drug according to claim 1, wherein:
the drug administration sheath is inserted into the body cavity until the second lumen reaches the deep part of the body cavity;
the drug supplied through the first lumen is retained in the body cavity and the balloon is immersed in the drug; and
the balloon is inflated, and the drug around the balloon is spread and applied onto the mucosa in the body cavity as the balloon inflates.

3. The method of applying a drug according to claim 1, wherein:
the drug administration sheath is inserted into the body cavity until the second lumen reaches the deep part of the body cavity;
the balloon is inflated; and
the drug is supplied through the first lumen into the body cavity, spread in a gap between the inflated balloon and the mucosa in the body cavity, and applied onto the mucosa.

4. The method of applying a drug according to claim 1, wherein:
a distal end of the drug administration sheath is inserted into the body cavity until the second lumen reaches the deep part of the body cavity;
the balloon is inflated to a specified size;
the drug supplied through the first lumen is retained in the body cavity and the balloon is immersed in the drug; and
the balloon is inflated again, and the drug around the balloon is spread and applied onto the mucosa in the body cavity as the balloon inflates.

5. The method of applying a drug according to claim 1, wherein:
the second lumen is provided with an index indicative of an amount of the drug administered; and
the drug administration sheath is inserted into the body cavity along with a soft endoscope to capture an image of the index, and when the drug supplied through the first lumen and retained in the body cavity reaches the index, supply of the drug is stopped.

6. The method of applying a drug according to claim 2, wherein:
the inflated balloon is moved from the deep part toward the ostium, while being maintained at a predetermined fixed pressure; and
the drug around the balloon is applied onto the wall portion of the body cavity from the deep part toward the ostium by the movement of the balloon.

7. The method of applying a drug according to claim 1, wherein the balloon is provided to cover all or part of a circumference of the drug administration sheath, and placed in one of
a first placement in which the distal end of the balloon almost coincides with the distal end of the drug administration sheath;
a second placement in which a proximal end of the balloon almost coincides with an ostium of the drug administration lumen, so that the balloon having an annular shape is attached to the ostium of the drug administration lumen; and
a third placement in which the balloon is placed in a portion of the drug administration lumen and spaced apart from the ostium of the drug administration sheath.
